(19) United States
(12) Patent Application Publication
(10) Pub. No.: US 2021/0077018 A1
(43) Pub. Date: Mar. 18, 2021

(54) METHOD AND SYSTEM TO SIMULTANEOUSLY PERFORM THERMOACOUSTIC AND ULTRASOUND IMAGING

(71) Applicant: ENDRA Life Sciences Inc., Ann Arbor, MI (US)

(72) Inventor: Jang Hwan Cho, Ann Arbor, MI (US)

(73) Assignee: ENDRA Life Sciences Inc., Ann Arbor, MI (US)

(21) Appl. No.: 16/568,997

(22) Filed: Sep. 12, 2019

(51) Int. Cl. A61B 5/00; A61B 5/05

(52) U.S. Cl. CPC A61B 5/4872; G01N 29/2431; A61B 5/05; A61B 5/0093

(57) ABSTRACT

A method and system for simultaneously performing ultrasound and thermoacoustic imaging includes directing RF energy with an RF emitter through a surface of the RF emitter and toward a region of interest within the object, wherein the region of interest has a material, a reference, and a boundary between the material and the reference, wherein the RF energy thermoacoustically induces an ultrasound signal at the surface of the RF emitter that travels through the object and reflects at the boundary; using the thermoacoustic system to receive a thermoacoustic multi-polar signal from a specific location on the boundary, wherein the thermoacoustic multi-polar signal is induced by the RF energy; receiving the reflected ultrasound signal; utilizing the thermoacoustic system and the reflected ultrasound signal to map the specific location within the object; and utilizing the thermoacoustic system and the thermoacoustic multi-polar signal to determine a parameter at the specific location.

### FIELD

This application relates to a method and system to simultaneously perform both ultrasound and thermoacoustic imaging.

### BACKGROUND

Currently, thermoacoustic imaging systems require a separate ultrasound system to identify the positions of relative imaging locations. This requires interoperability between two systems, creating the potential for error and complication.

Hence, it's desirable to add ultrasound imaging capability to a thermoacoustic system without implementing additional ultrasound transmission functionality. Ideally, both the thermoacoustic and ultrasound images would be acquired simultaneously with the same burst of radio-frequency (RF) energy.

A potential solution is described in Microwave-excited ultrasound and thermoacoustic dual imaging by Ding et al (available on May 5, 2017), Applied Physics Letters 110, 183701; https://aip.scitation.org/doi/10.1063/1.4983166 (herein referred to as Ding), which is incorporated by reference in its entirety.

The method described in Ding requires very high power (40 MW was used). Furthermore, transducer relaxation time, which determines how close an object can be from a transducer, can be very long for such a high power radio-frequency (RF) transmission. As a result, the long transducer relaxation time limits the field of view of the image.

Ding's method also generates the ultrasound by exciting the transducer with the RF energy. Most commercial ultrasound systems and transducers are designed to minimize RF interference (RFI). Directing RF energy to the transducer without affecting the system is not trivial. Enough energy needs to be delivered to the piezoelectric element of the transducer, but at the same time, such RFI should be avoided to protect the system. To properly perform such a method, a specially-designed ultrasound transducer or ultrasound system is required.

Furthermore, to properly direct the RF energy into the transducer, the RF applicator should be directed toward the transducer. This implies that the RF applicator should be on the other side of the object or body to be measured. This further complicates the excitation of the transducer because the energy delivered to the transducer is affected by the object. This poses strong physical constraints for the system.

Hence, there exists an unmet need for an integrated system that combines ultrasound and thermoacoustic imaging in a novel way, without the limitations cited in Ding.

### SUMMARY

Described herein are methods and systems for a thermoacoustic imaging system that combines thermoacoustic imaging with ultrasound imaging from an ultrasound signal generated at an RF emitter surface. The ultrasound signal travels in an object and reflects at boundaries, thereby appearing after the thermoacoustic signal.

In one embodiment, a method for simultaneously performing ultrasound and thermoacoustic imaging comprises directing, by an imaging system, RF energy with an RF emitter through a surface of the RF emitter and toward a region of interest within the object, wherein the region of interest has a material, a reference, and a boundary between the material and the reference, further wherein the RF energy thermoacoustically induces an ultrasound signal at the surface of the RF emitter that travels through the object and reflects at the boundary; receiving, by a thermoacoustic system of the imaging system, a thermoacoustic multi-polar signal from a specific location on the boundary, wherein the thermoacoustic multi-polar signal is induced by the RF energy; receiving, by the thermoacoustic system of the imaging system, the reflected ultrasound signal; mapping, by a computing device of the imaging system, the specific location within the object based on the reflected ultrasound signal; and determining, by the computing device of the imaging system, a parameter of the object at the specific location based on the thermoacoustic multi-polar signal.

In a separate embodiment, the thermoacoustic system comprises one thermoacoustic transducer.

In a separate embodiment, the thermoacoustic system comprises a plurality of thermoacoustic transducers.

In a separate embodiment, the object is a human body.

In a separate embodiment, a parameter can be a fat concentration in tissue. In a separate embodiment, a parameter can be a conductivity of a blood vessel. In a separate embodiment, a parameter can be determined in a non-human scan and the parameter can be a Grüneisen parameter, conductivity, or the like.

In one embodiment, a thermoacoustic system configured to simultaneously performing ultrasound and thermoacoustic imaging comprises an RF emitter configured to direct RF energy toward a region of interest within an object, wherein the region of interest has a material, a reference, and a boundary between the material and the reference, wherein the RF energy induces a thermoacoustic multi-polar signal from the boundary and an ultrasound signal from the boundary; at least one thermoacoustic transducer configured to receive the thermoacoustic multi-polar signal and the ultrasound signal; and a processor configured to receive the ultrasound signal from the at least one thermoacoustic transducer and determine a specific location within the object, and the processor is configured to receive the thermoacoustic multi-polar signal from the at least one thermoacoustic transducer to determine a parameter at the specific location.

In yet another embodiment, an imaging system for simultaneously performing ultrasound and thermoacoustic imaging comprises an RF emitter configured to direct RF energy to a region of interest within an object, wherein the region of interest has a material, a reference, and a boundary between the material and the reference, further wherein the RF energy thermoacoustically induces an ultrasound signal at the surface of the RF emitter that travels through the object and reflects at the boundary; a thermoacoustic transducer array configured to receive a thermoacoustic multi-polar signal from the specific location on the boundary, wherein the thermoacoustic multi-polar signal is induced by the RF energy, and receive the reflected ultrasound signal; and a computing device configured to map the specific location within the object based on the reflected ultrasound signal, and determine a parameter of the object at the specific location based on the thermoacoustic multi-polar signal.

### BRIEF DESCRIPTION OF THE DRAWINGS

Embodiments will now be described more fully with reference to the accompanying drawings in which:

FIG. 1A shows an imaging system in relation to a target;

FIG. 1B shows an imaging system in relation to a target with reference padding;

FIG. 1C shows an imaging system in relation to a target with reference padding and two separate boundaries;

FIG. 1D shows an imaging system with an RF matching layer in relation to a target with reference padding;

FIG. 2 shows exemplary multi-polar signals;

FIG. 3 is a graph showing exemplary electric field strength attenuation curves;

FIG. 4 shows electric field strength attenuation curves in tissue as a function of distance from the RF source of the thermoacoustic imaging system;

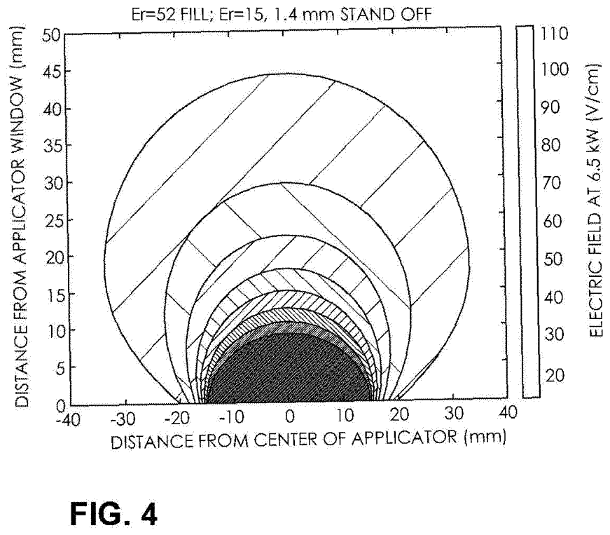

FIG. 5 shows exemplary multi-polar signals with an intermediate material or tissue between the reference and object of interest;

FIG. 6 is a flowchart of a method to simultaneously perform thermoacoustic and ultrasound imaging.

### DETAILED DESCRIPTION OF THE EMBODIMENTS

The present disclosure discusses methods and systems that combine ultrasound and thermoacoustic imaging. The methods and systems enable simultaneous thermoacoustic and ultrasonic imaging of an object for a thermoacoustic imaging system that does not have ultrasound transmission capability.

The methods and systems described herein do not use ultrasound generated from the transducer via RF excitation. Instead the ultrasound signal is generated at the RF applicator surface.

In various embodiments, an ultrasonic wave is generated at the applicator surface to obtain ultrasound imaging in the thermoacoustic imaging system, with the same RF radiation dose and without any additional system modification.

In various embodiments, pads can further separate thermoacoustic and ultrasound imaging.

In various embodiments, the methods and systems adjust RF power based on the pad and desired signal characteristics.

The thermoacoustic transducer can be shielded to minimize the effect of radio frequency interference (RFI). Hence, any ultrasound signal generated from residual RFI will be small compared to an actual thermoacoustic signal, especially when it is reflected off a boundary within an object that is being imaged.

Because the electric field is very high at the applicator surface and there will be a mismatch in dielectric properties between the matching layer of the applicator (which is at the applicator surface) and the object, a strong thermoacoustic signal will be generated at the applicator surface. This ultrasound signal travels in the object and reflects at boundaries. Due to its strong intensity, the reflected signal is still large enough to be detected. Due to longer travel time, the reflected signal appears after the thermoacoustic signal. Using a time separation technique, each portion can be used to generate thermoacoustic and ultrasound images, respectively.

The methods and systems of the present disclosure can have a limited field of view. This can be detrimental. For example, if there is a boundary near the applicator, a reflection off the boundary is likely to interfere with thermoacoustic signals from other boundaries.

One potential solution to this limited field of view is to use a pad in front of the probe. The pad functions as an intermediate layer and creates an offset, which widens the field of view. With the pad, the first possible reflected ultrasound signal would arrive at the transducer with a delay that is a function of twice the thickness of the pad. Using enough thickness for the pad ensures that the thermoacoustic signals do not interfere with reflected signals.

The pad will attenuate transmitted ultrasound, so the pad should use a low loss material of construction. In one embodiment, the density of the pad provides an attenuation loss of signal that matches the attenuation loss that occurs in human tissue. The match can be to fat, muscle, bone, or some combination thereof. The match will be within a predetermined tolerance, such as 10%.

Also, the pad should provide good impedance matching to ensure good RF energy delivery. The impedance match maximizes RF transmission into the object of interest (such as a human body). Impedance matching (i.e., utilizing an impedance which matches an impedance found in a human body) can minimize signal reflection (resulting in greater energy transmission into the object of interest).

Different pad thicknesses will be used, based on desired signal separation. For different pad thicknesses, the input RF power can also be adjusted to compensate for the forward power loss that is due to the pad. The methods and systems can automatically determine the optimal power when the known pad is attached. Furthermore, the transducer relaxation time will also be considered to determine the optimal RF power. Hence, the method and system can optimize RF power according to a given pad thickness and desired transducer relaxation.

The ultrasound image is registered with the thermoacoustic image so that the locations of both images match. Because the ultrasound image is generated from a reflected signal, angle compensation may be required.

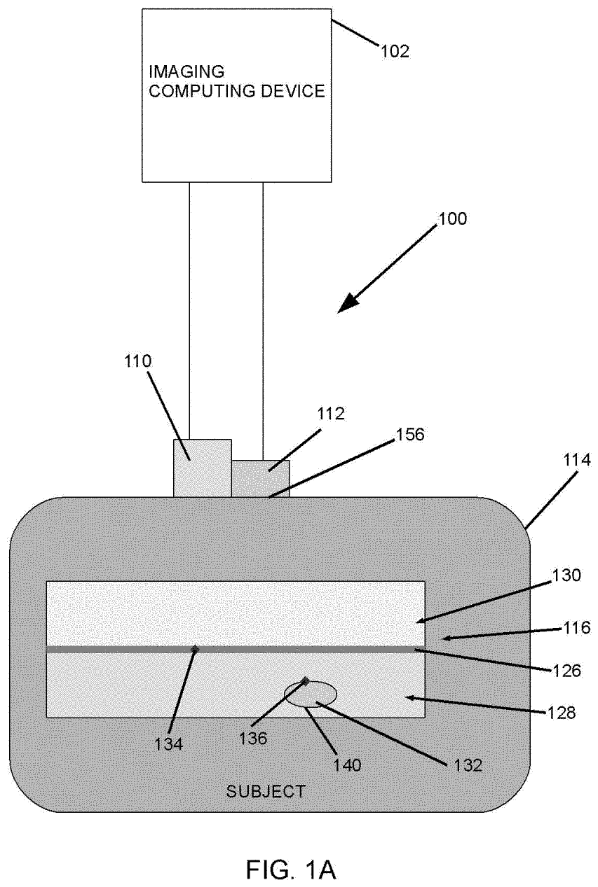

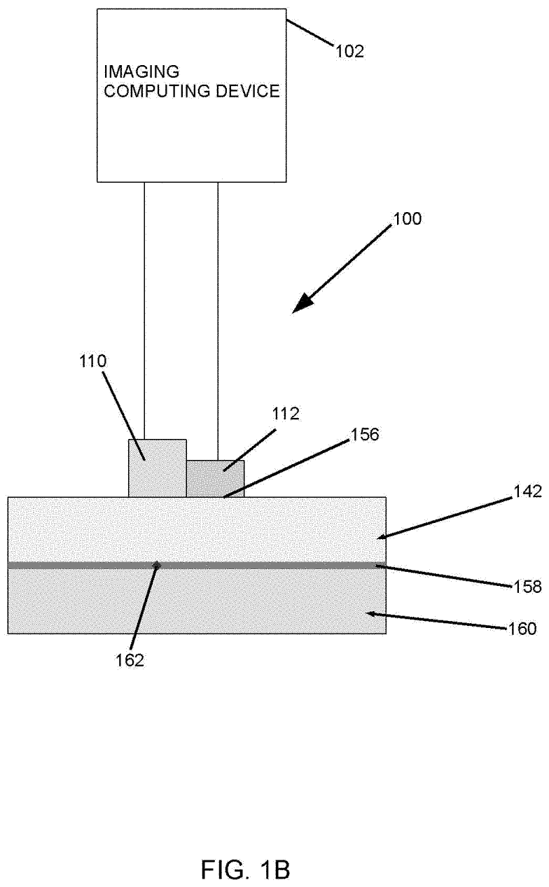

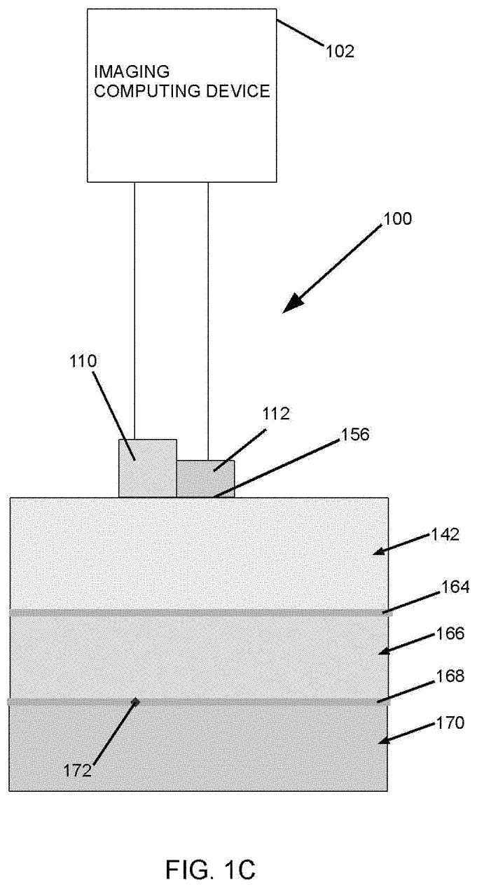

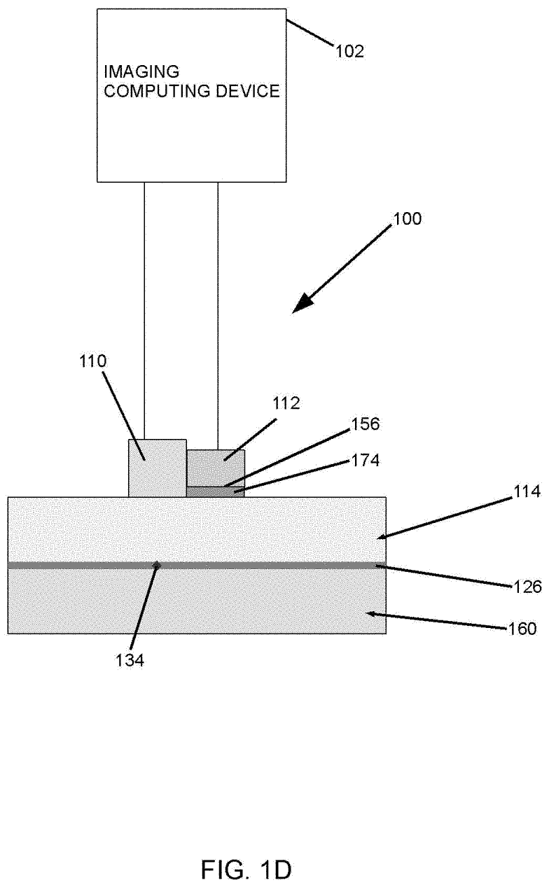

Turning now to FIG. 1A, an imaging system 100 is shown in relation to a target 114. In this embodiment, the imaging system 100 comprises an imaging computing device 102 communicatively coupled to one or more thermoacoustic transducer arrays 110 and a radio-frequency (RF) emitter 112. Imaging system 100 is configured to obtain ultrasound and thermoacoustic data respectively of a region of interest 116 associated within a target 114.

In one embodiment, the target 114 is an inanimate object. In a separate embodiment, the target 114 is a human body. In a separate embodiment, the target 114 is an animal.

In the embodiment depicted in FIG. 1A, the region of interest 116 comprises first reference 130, first boundary 126, second boundary 140, first boundary location 134, second boundary location 136, object of interest 128, and second object of interest (e.g., tumor) 132.

The imaging computing device 102 in this embodiment is a machine comprising a personal computer or other suitable processing device comprising, for example, a processing unit comprising one or more processors, non-transitory system memory (volatile and/or non-volatile memory), other non-removable or removable memory (e.g., a hard disk drive, RAM, ROM, EEPROM, CD-ROM, DVD, flash memory, etc.) and a system bus coupling the various computer components to the processing unit. The imaging computing device 102 may also comprise networking capabilities using Ethernet, Wi-Fi, and/or other suitable network format, to enable connection to shared or remote drives, one or more networked computers, or other networked devices. One or more input devices, such as a mouse, stylus, touchscreen, and/or a keyboard (not shown) are coupled to the imaging computing device 102 for receiving user input. A display device (not shown), such as a computer screen or monitor, is coupled to the imaging computing device 102 for displaying one or more generated images that are based on ultrasound image data and thermoacoustic image data received from imaging computing device 102.

In one embodiment, the RF emitter 112 has a frequency between about 10 MHz and 100 GHz and has a pulse duration between about 0.1 nanoseconds and 10 microseconds. Acoustic pressure waves detected by the one or more thermoacoustic transducer arrays 110 are processed and communicated as thermoacoustic data and ultrasound data to the imaging computing device 102 for further processing and for presentation and interpretation by an operator.

The imaging computing device 102 initiates the RF emitter 112 to send RF energy through RF emitter surface 156 into the target 114. Some of the RF energy is absorbed at the RF emitter surface 156. The RF energy that is absorbed at the RF emitter surface 156 creates an ultrasound pulse that travels into the target 114.

The RF energy that is not absorbed at the RF emitter surface 156 or reflected from the target 114 is absorbed by the target 114. The RF energy then travels to and is absorbed in the region of interest 116. Within the region of interest 116, there is first boundary 126 between reference 130 and an object of interest 128. There is also a second boundary 140 between object of interest 128 and second object of interest 132. The difference between RF energy absorbed in reference 130 and object of interest 128 creates a thermoacoustic multi-polar signal emanating from boundary location 134. The difference between RF energy absorbed by object of interest 128 and second object of interest 132 creates a second thermoacoustic multi-polar signal emanating from boundary location 136. Thermoacoustic transducer array 110 receives the thermoacoustic multi-polar signals and sends the resulting data to the thermoacoustic imaging computing device 102.

Similarly, the ultrasound pulse that is created at the RF emitter surface 156 is absorbed into target 114 and reflects off of objects within the target 114. The ultrasound pulse travels into the region of interest 116, reflects at boundaries 126 and 140 and at locations 134 and 136, and then travels back to thermoacoustic transducer array 110, where the ultrasound signals are interpreted by the imaging system 100.

Turning now to FIG. 1B, a separate embodiment is shown with some of the same elements as FIG. 1A. In addition, FIG. 1B shows that the thermoacoustic transducer arrays 110 and RF emitter 112 directly contact padding (or padding material) 142, which serves the same purpose and function as reference 130. Boundary 158 lies between padding 142 and target region 160. FIG. 1B shows an embodiment where the method is performed on an inanimate target.

The imaging computing device 102 initiates the RF emitter 112 to send RF energy through RF emitter surface 156 into the padding 142. Some of the RF energy is absorbed at the RF emitter surface 156. The RF energy that is absorbed at the RF emitter surface 156 creates an ultrasound pulse that travels through the padding 142. The RF energy then travels to boundary 158 between padding 142 and target region 160. The difference between RF energy absorbed in padding 142 and target region 160 creates a thermoacoustic multi-polar signal emanating from boundary location 162. Thermoacoustic transducer array 110 receives the thermoacoustic multi-polar signal emanating from boundary location 162 and sends the resulting data to the thermoacoustic imaging computing device 102.

Similarly, the ultrasound pulse that is created at the RF emitter surface 156 is travels through padding 142 and reflects off the boundary 158 at location 162. The ultrasound signals reflected at location 162 then travel back to thermoacoustic transducer array 110, where the ultrasound signals are interpreted by the imaging system 100.

FIG. 1C shows an imaging system in relation to a target with reference padding and two separate boundaries. The imaging computing device 102 initiates the RF emitter 112 to send RF energy through RF emitter surface 156 into the padding 142. Some of the RF energy is absorbed at the RF emitter surface 156. The RF energy that is absorbed at the RF emitter surface 156 creates an ultrasound pulse that travels through the padding 142. The RF energy travels to first material boundary 164 between padding 142 and first material 166, then to second material boundary 168 between first material 166 and second material 170. The difference between RF energy absorbed in first material 166 and second material 170 creates a thermoacoustic multi-polar signal emanating from second material boundary location 172. Thermoacoustic transducer array 110 receives the thermoacoustic multi-polar signal emanating from boundary location 172 and sends the resulting data to the thermoacoustic imaging computing device 102.

Similarly, the ultrasound pulse that is created at the RF emitter surface 156 travels through padding 142, first material 166, and then reflects from the second material boundary at location 172. The ultrasound signals reflected at location 172 then travel back to thermoacoustic transducer array 110, where the ultrasound signals are interpreted by the imaging system 100.

FIG. 1D shows an imaging system with an RF matching layer in relation to a target with reference padding. Matching layer 174 is shown affixed to RF emitter surface 156. The other elements are shown as described in FIG. 1B. The matching layer 174 is a thin layer that serves to adjust the RF signal that is received at the surface of a target 114.

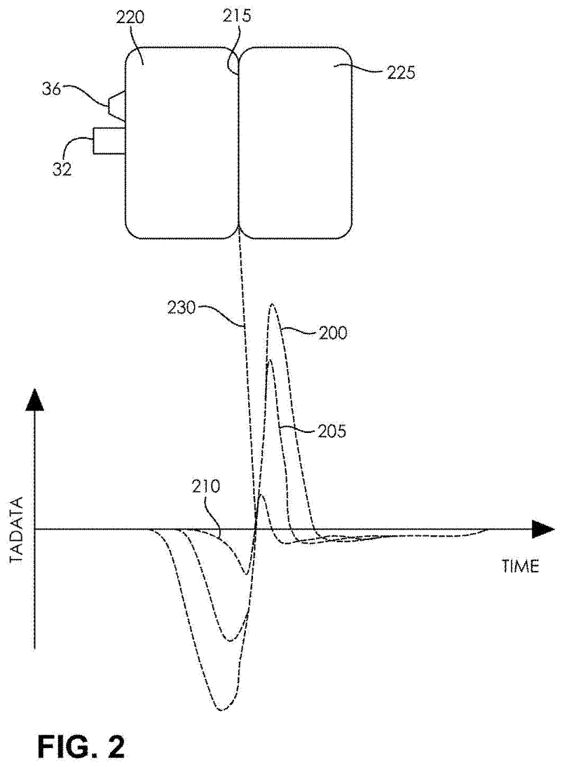

Exemplary multi-polar acoustic signals 200, 205 and 210 are shown in FIG. 2. The system includes a thermoacoustic transducer 32 (described above) and an RF emitter 36 that directs an energy signal through a first tissue 220. The multi-polar acoustic signals 200, 205 and 210 are generated in response to thermoacoustic imaging of a tissue region of interest (ROI) comprising the first tissue 220 and a different type of second tissue 225 that are separated by a boundary 215. A dashed line 230 indicates a time point corresponding to the boundary 215. The differences in the peak-to-peak values of the multi-polar acoustic signals 200, 205 and 210 represent the extent to which the first tissue 220 expands into the boundary 215 and into the second tissue 225 before contracting. As the difference between the amount of energy absorbed of the two different tissues at the boundary 215 increases, the amount that the first tissue 220 expands into the boundary 215 and into the second tissue 225 increases. Therefore, the peak-to-peak amplitude of each multi-polar acoustic signal 200, 205 and 210 is proportional to the difference between the amount of energy absorbed of the two different tissues at the boundary 215. As can be seen, the peak-to-peak value of multi-polar acoustic signal 200 is greater than that of multi-polar acoustic signals 205, 210, and the peak-to-peak value of multi-polar acoustic signal 205 is greater than that of multi-polar acoustic signal 210. As such, the difference between the amount of energy absorbed of the two different tissues at the boundary 215 when multi-polar acoustic signal 200 is generated is greater than the difference between the amount of energy absorbed of the two different tissues at the boundary 215 when multi-polar signal 205 is generated. Similarly, the difference between the amount of energy absorbed of the two different tissues at the boundary 215 when multi-polar acoustic signal 205 is generated is greater than the difference between the amount of energy absorbed of the two different tissues at the boundary 215 when multi-polar signal 210 is generated.

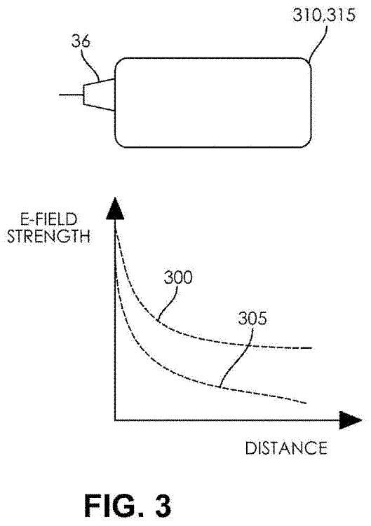

FIG. 3 shows electric field strength attenuation curves 300, 305 in material 310, 315 as a function of distance from an RF emitter (applicator) 36 of a thermoacoustic imaging system. The example is simplified and ignores factors such as reflections off an object boundary. Each electric field strength attenuation curve 300, 305 represents the electric field strength attenuation of material 310, 315, respectively, as a function of distance from the RF applicator 36. Each electric field strength attenuation curve 300, 305 corresponds to a material, each of which has a different attenuation coefficient (which could correspond to a different fat concentration for each respective material). The material associated with electric field strength curve 300 has a lower attenuation coefficient than the material associated with electric field strength curve 305. In one embodiment, the material with a lower attenuation coefficient is has a high fat concentration (e.g., greater than 10%) and the material with a higher attenuation coefficient has a low fat concentration (e.g., less than 10%).

The material 310 associated with electric field strength attenuation curve 300 has a different Grüneisen parameter than the material 315 associated with electric field strength attenuation curve 305.

Different materials (e.g., tissues) have characteristic dielectric properties at a given frequency and a temperature. The dielectric properties of a material determines how much energy is absorbed by the material. An electric field transmitted through the material is attenuated, and the amount of attenuation is determined by both dielectric and physical properties of the material. As an example, compared to normal tissue, fatty tissue absorbs less energy and thus attenuates less electric field. Knowing these properties, the amount of attenuation through a material can be estimated. Furthermore, for a given RF applicator with specific design and tuning, dielectric properties of a material lead to different RF matching and energy delivery. For example, if the RF applicator is tuned to match well on human body, it is likely to match poorly to material with high water content, such as ultrasound gel. Therefore, knowing the RF power and matching properties gives information on the material in contact with the applicator.

Although in embodiments described above the boundary is selected at a location where the target material and the reference material are in close relation to one another, those skilled in the art will appreciate that alternatives are available. For example, in another embodiment, an intermediate structure may be in between the reference material and the target material.

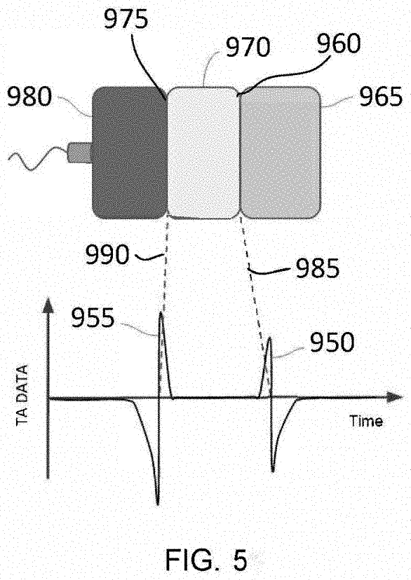

Example multi-polar signals 950 and 955 of this embodiment are shown in FIG. 5. The multi-polar signals 950 and 955 represent thermoacoustic data obtained at a boundary 960 between reference material 965 and intermediate material 970 and a boundary 975 between intermediate material 970 and target material 980, respectively. The dashed line 985 indicates a time point corresponding to the boundary 960, and dashed line 990 indicates a time point corresponding to the boundary 975.

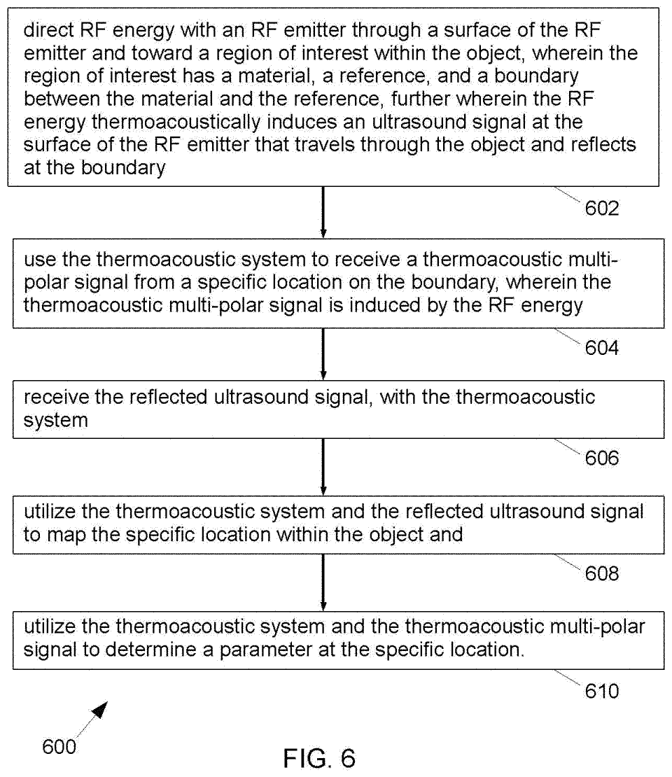

FIG. 6 is a flowchart of a method for calculating a material parameter of an object of interest 600. Shown are the steps of directing RF energy with an RF emitter through a surface of the RF emitter and toward a region of interest within the object, wherein the region of interest has a material, a reference, and a boundary between the material and the reference, further wherein the RF energy thermoacoustically induces an ultrasound signal at the surface of the RF emitter that travels through the object and reflects at the boundary 602; using the thermoacoustic system to receive a thermoacoustic multi-polar signal from a specific location on the boundary, wherein the thermoacoustic multi-polar signal is induced by the RF energy 604; receiving the reflected ultrasound signal, with the thermoacoustic system 606; utilizing the thermoacoustic system and the reflected ultrasound signal to map the specific location within the object 608; and utilizing the thermoacoustic system and the thermoacoustic multi-polar signal to determine a parameter at the specific location 610. Note that step 608 performs the mapping in a way similar to a conventional ultrasound imaging system.

Although embodiments have been described above with reference to the accompanying drawings, those of skill in the art will appreciate that variations and modifications may be made without departing from the scope thereof as defined by the appended claims.

What is claimed is:

1. A method for simultaneously performing ultrasound and thermoacoustic imaging, the method comprising:
directing, by an imaging system, RF energy with an RF emitter through a surface of the RF emitter and toward a region of interest within the object, wherein the region of interest has a material, a reference, and a boundary between the material and the reference, further wherein the RF energy thermoacoustically induces an ultrasound signal at the surface of the RF emitter that travels through the object and reflects at the boundary;
receiving, by a thermoacoustic system of the imaging system, a thermoacoustic multi-polar signal from a specific location on the boundary, wherein the thermoacoustic multi-polar signal is induced by the RF energy;
receiving, by the thermoacoustic system of the imaging system, the reflected ultrasound signal;
mapping, by a computing device of the imaging system, the specific location within the object based on the reflected ultrasound signal; and
determining, by the computing device of the imaging system, a parameter of the object at the specific location based on the thermoacoustic multi-polar signal.

2. The method of claim 1, wherein the thermoacoustic system comprises one thermoacoustic transducer.

3. The method of claim 1, wherein the thermoacoustic system comprises a plurality of thermoacoustic transducers.

4. The method of claim 1, wherein the object is a human body.

5. The method of claim 1, wherein the parameter is a fat concentration.

6. The method of claim 1, wherein determining the parameter of the object comprises determining an attenuation of an electric field within the object.

7. The method of claim 6, wherein determining the parameter of the object further comprises comparing the attenuation of the electric field within the object to a known attenuation of an electric field within a known material.

8. A thermoacoustic system configured to simultaneously performing ultrasound and thermoacoustic imaging, the system comprising:
an RF emitter configured to direct RF energy toward a region of interest within an object, wherein the region of interest has a material, a reference, and a boundary between the material and the reference, wherein the RF energy induces a thermoacoustic multi-polar signal from the boundary and an ultrasound signal from the boundary;
at least one thermoacoustic transducer configured to receive the thermoacoustic multi-polar signal and the ultrasound signal; and
a processor configured to receive the ultrasound signal from the at least one thermoacoustic transducer and determine a specific location within the object, and the processor is configured to receive the thermoacoustic multi-polar signal from the at least one thermoacoustic transducer to determine a parameter at the specific location.

9. The thermoacoustic system of claim 8, wherein the thermoacoustic system comprises one thermoacoustic transducer.

10. The thermoacoustic system of claim 8, wherein the thermoacoustic system comprises a plurality of thermoacoustic transducers.

11. The thermoacoustic system of claim 8, wherein the object is a human body.

12. The thermoacoustic system of claim 8, wherein the parameter is a fat concentration.

13. The thermoacoustic system of claim 8, wherein determining the parameter of the object comprises determining an attenuation of an electric field within the object.

14. The thermoacoustic system of claim 13, wherein determining the parameter of the object further comprises comparing the attenuation of the electric field within the object to a known attenuation of an electric field within a known material.

15. An imaging system for simultaneously performing ultrasound and thermoacoustic imaging, the imaging system comprising:
an RF emitter configured to direct RF energy to a region of interest within an object, wherein the region of interest has a material, a reference, and a boundary between the material and the reference, further wherein the RF energy thermoacoustically induces an ultrasound signal at the surface of the RF emitter that travels through the object and reflects at the boundary;
a thermoacoustic transducer array configured to receive a thermoacoustic multi-polar signal from the specific location on the boundary, wherein the thermoacoustic multi-polar signal is induced by the RF energy, and receive the reflected ultrasound signal; and
a computing device configured to map the specific location within the object based on the reflected ultrasound signal, and determine a parameter of the object at the specific location based on the thermoacoustic multi-polar signal.

16. The imaging system of claim 15, wherein the parameter is a fat concentration.

17. The imaging system of claim 15, wherein the thermoacoustic transducer array comprises a plurality of thermoacoustic transducers.

18. The imaging system of claim 15, wherein the object is a human body.

19. The imaging system of claim 15, wherein determining the parameter of the object comprises determining an attenuation of an electric field within the object.

20. The imaging system of claim 19, wherein determining the parameter of the object further comprises comparing the attenuation of the electric field within the object to a known attenuation of an electric field within a known material.
